(19) United States
(12) Patent Application Publication
(10) Pub. No.: US 2019/0137382 A1
(43) Pub. Date: May 9, 2019

(54) Monitoring Contamination in a Stream of Fiber Flocks

(71) Applicant: Uster Technologies AG, Uster (CH)

(72) Inventors: Thomas Nasiou, Uster (CH); Sivakumar Narayanan, Uster (CH); Elvis Kaljic, St. Gallen (CH)

(73) Assignee: Uster Technologies AG, Uster (CH)

(21) Appl. No.: 16/094,266

(22) PCT Filed: Apr. 28, 2017
(86) PCT No.: PCT/CH2017/000040

§ 371 Date: Oct. 17, 2018

(30) Foreign Application Priority Data
May 4, 2016 (CH) 00589/16

(51) Int. Cl. G01N 15/14; D01G 23/08; G01N 21/85; G01N 21/94; G01N 33/36; G01N 21/89

(52) U.S. Cl. CPC G01N 15/1459; D01G 23/08; G01N 21/85; G01N 21/94; G01N 2021/8592; G01N 21/8915; G01N 15/1463; G01N 2015/1493; G01N 33/362

(57) ABSTRACT

The method is for monitoring contamination in a stream of fiber flocks transported pneumatically in an airflow. Characteristics of entities, including contamination, in the stream of fiber flocks are detected and evaluated. Values of a first parameter and a second parameter of the entities are determined from the characteristics of the entities. An event field is provided, which contains a quadrant or a part of a quadrant of a two-dimensional Cartesian coordinate system, wherein a first axis defines the first parameter and a second axis defines the second parameter. The values of the first parameter and the second parameter determined for an entity are entered in the event field as coordinates of an event representing the entity. Thus, entities can be handled in a differentiated way.

### BACKGROUND OF THE INVENTION

The invention relates to a method and a device for monitoring contamination in a stream of fiber flocks, according to the preambles of the independent claims. Its preferred application is in spinning preparation, and in particular in monitoring raw cotton fibers in the blow room.

### DESCRIPTION OF THE PRIOR ART

WO-2006/079426 A1 discloses a method and device for removing foreign matters from a fiber material, in particular from raw cotton. Such methods are used for example in the blow room to prepare the raw cotton for the spinning process. Here, at the earliest possible time after opening the cotton bales, foreign fiber such as cords, jute shreds, plastic films, and the like are removed. The raw cotton is conveyed in a pneumatic fiber conveying conduit past a sensor system and a separating device. The sensor system consists of two cameras. Upon detection of foreign matters by the sensor system, the foreign matters are removed by a pulse of compressed air transverse to the fiber conveying direction via a removal opening in the fiber conveying conduit.

While the above removing method is performed on-line, during production, U.S. Pat. No. 5,539,515 A relates to a laboratory, i.e., off-line measurement. It discloses an apparatus and method for measurement and classification of trash in fiber samples. A fiber sample is provided to a processor, where entities are individualized and thereafter transported to a sensor system. Characteristic signals are generated by the sensor signal corresponding to sensed characteristics of the entities, including trash. A computer analyzes the characteristic signals to identify signals corresponding to trash and to classify the trash signals as corresponding to one of several types of trash. Based on the characteristic signals, the computer determines an entity length, diameter and speed and also determines a peak value of a characteristic signal corresponding to an entity. Based on these measurements, trash is characterized as to one of several types of trash.

A process and device for monitoring the quality of yarns are disclosed in U.S. Pat. No. 6,244,030 B1. In order to differentiate extraneous materials in a yarn section from the yarn itself and from other extraneous materials, a signal derived from the yarn is classified in a classification field. The classification field or classification matrix has a horizontal axis along which the length of the extraneous materials is plotted, and a vertical axis along which the reflectivity of the extraneous materials is plotted. On the basis of that classification, the extraneous materials contained in the yarn and their types can be determined.

For ensuring the quality in yarn production, so-called yarn clearers are used in spinning or winding machines. A yarn clearer contains a measuring head with at least one sensor which scans the moved yarn and detects defects such as thick places, thin places or foreign matter in the yarn. The output signal of the sensor is continuously evaluated according to predetermined evaluation criteria. The evaluation criteria are predetermined by a clearing limit in form of a clearing curve in a two-dimensional classification field or event field which is established by the length of the event on the one hand and an amplitude of the event on the other hand, e.g. a deviation of the yarn mass from a reference value. Events beneath the clearing curve will be tolerated; whereas events above the clearing curve will be removed from the yarn or at least registered as defects. An example of an event field with a clearing curve is shown in U.S. Pat. No. 6,374,152 B1.

A clearing curve in a two-dimensional classification field for yarn defects is also shown in EP-2,644,553 A2. The horizontal axis of the classification field designates the yarn-defect length and the vertical axis designates the yarn-defect thickness.

WO-2011/038524 A1 discloses a method for setting a clearing limit on an electronic yarn clearing system. First, a statistical representation of the test material is determined by means of measurements of the test material. Based on the statistical representation, a clearing limit is calculated and proposed for use, wherein a length-related number of impermissible events to be expected with said clearing limit is calculated and output. An operator can provide a comment on the number of impermissible events to be expected, whereupon the clearing limit is automatically set according to the comment.

### SUMMARY OF THE INVENTION

It is an object of the invention to further improve the monitoring of contamination in a stream of fiber flocks. It is a further object to provide an objective basis for the monitoring of contamination in a stream of fiber flocks and thus make the monitoring results from various monitoring locations and times comparable with each other. It is still a further object to provide a more complete and more accurate definition of the contamination to be removed from the stream of fiber flocks.

These and other objects are solved by the method and device defined in the independent claims. Advantageous embodiments are indicated in the dependent claims.

The invention is based on the idea of providing a two-dimensional “event field” for entities in the stream of fiber flocks. The event field is defined by two axes representing two parameters determined from characteristics of the entities. The values of the parameters are entered in the event field as coordinates of an event representing the entity. Various classes of entities can be defined in the event field, and an entity can be classified in one of the classes. By counting the entities classified in the various classes, the stream of fiber flocks can be characterized by the individual numbers of entities counted in each class. Such classifications can be provided for several types of contaminants separately. The various types of contaminants can be distinguished from each other by means of a third parameter determined from characteristics of the entities. Moreover, a removal limit in form of a removal curve in the event field can be predetermined as a criterion for the permissibility or impermissibility of the entities.

The term “entity” as used throughout the present document designates a component of a fiber flock. An entity can be, for instance, a fiber constituting the base material, a bundle of fibers (mechanical nep), a fragment of a seed coat, leaf or stem (biological nep), a permissible or an impermissible contamination.

In the method according to the invention for monitoring contamination in a stream of fiber flocks transported pneumatically in an airflow, characteristics of entities, including contamination, in the stream of fiber flocks are detected and evaluated. Values of a first parameter and a second parameter of the entities are determined from the characteristics of the entities. An event field is provided, which contains a quadrant or a part of a quadrant of a two-dimensional Cartesian coordinate system, wherein a first axis defines the first parameter and a second axis defines the second parameter. The values of the first parameter and the second parameter determined for an entity are entered in the event field as coordinates of an event representing the entity.

The event field, including a scatter plot showing the coordinates of events representing entities, is preferably graphically represented.

The first parameter can be, for example, related to a geometric characteristic of the entities, and preferably is a length or an area of the entities.

The second parameter can be, for example, related to an optical characteristic of the entities, and preferably is an intensity of electromagnetic radiation after interaction with the entities.

According to one embodiment, at least two classes of entities in form of non-overlapping areas in the event field are predetermined, and an entity is classified in one of the at least two classes when the coordinates of an event representing the entity lie in the corresponding area.

The event field including the at least two areas is preferably graphically represented.

The areas can be, for example, adjacent rectangles delimited from each other by straight lines parallel to the first axis or to the second axis.

It is advantageous to count entities classified in at least one of the at least two classes and to output for each of the at least one of the at least two classes the individual numbers of entities counted.

In one embodiment, at least two event fields are provided, each of the at least two event fields containing a quadrant or a part of a quadrant of a two-dimensional Cartesian coordinate system, wherein a first axis defines the first parameter and a second axis defines the second parameter. In each of the at least two event fields at least two classes of entities in form of non-overlapping areas in the event field are predetermined. A criterion related to at least a third parameter of entities is assigned to each of the at least two event fields. Values of the least a third parameter are determined from the characteristics of the entities. An entity is classified in one of the at least two event fields, depending on the fulfillment of the criterion by the value of the at least third parameter determined for said entity.

The third parameter can be, for example, related to an optical characteristic of the entities, and preferably is a spectral distribution of broadband electromagnetic radiation after interaction with the entities.

According to one embodiment, a removal limit in form of a removal curve in the event field is predetermined as a criterion for the permissibility or impermissibility of the entities. Entities with coordinates on the one side of the removal curve are left in the stream of fiber flocks, whereas entities with coordinates on the other side of the removal curve are removed from the stream of fiber flocks.

The event field including the removal curve is preferably graphically represented.

According to one embodiment, a statistical representation of the stream of fiber flocks is determined from the values of the first parameter and the second parameter of the entities. The removal limit is predetermined on the basis of the statistical representation. A time-related or mass-related number of impermissible entities is calculated from the established statistical representation and the removal limit. The time-related or mass-related number of impermissible entities is output on an output unit. An operator is requested to enter a comment on the output time-related or mass-related number of impermissible entities by means of an input unit. The removal limit is set automatically according to the entered comment.

As a result of the comment, the operator can decide whether the proposed removal limit is sufficient for the desired application, or whether it needs to be tightened or loosened. “Tightened” shall mean in this case that more events are to be removed; “loosened” shall mean that fewer events are to be removed. The system then performs corrections on the removal limit which lead to the desired behavior. In order to make the settings as simple as possible for the operator, the operator is offered the possibility to increase or decrease the number of impermissible entities by an incremental value by way of a simple mouse click or by pressing a button.

According to one embodiment, a second monitoring of contamination is subsequently performed in the stream of fiber flocks, or in an intermediate or a product containing fibers from the stream of fiber flocks, and the removal limit in the monitoring of contamination in the stream of fiber flocks is changed depending on a result of the second monitoring of contamination. Thus, there is a closed control loop controlling the removal of contamination in the stream of fiber flocks.

According to one embodiment, a second monitoring of contamination is subsequently performed in the stream of fiber flocks, or in an intermediate or a product containing fibers from the stream of fiber flocks, and a removal limit in the second monitoring of contamination is changed depending on a result of the monitoring of contamination in the stream of fiber flocks. Thus, there is an open control loop controlling the removal of contamination in the stream of fiber flocks.

The second monitoring is preferably performed by means of a yarn clearer in a yarn containing fibers from the stream of fiber flocks.

The device according to the invention for monitoring contamination in a stream of fiber flocks comprises a pneumatic fiber transport conduit for transporting the stream of fiber flocks, a sensor system for detecting characteristics of entities, including contamination, in the stream of fiber flocks, the sensor system being arranged on the pneumatic fiber transport conduit, and an evaluation unit for evaluating output signals of the sensor system. The evaluation unit is configured for determining from the output signals of the sensor system values of a first parameter and a second parameter of the entities, providing an event field, which contains a quadrant or a part of a quadrant of a two-dimensional Cartesian coordinate system, wherein a first axis defines the first parameter and a second axis defines the second parameter, and entering the values of the first parameter and the second parameter determined for an entity in the event field as coordinates of an event representing the entity.

The device can further comprise an output unit for outputting a result of the evaluation, the output unit being configured for outputting a graphical representation of the event field, including a scatter plot showing the coordinates of events representing entities.

The sensor system preferably comprises a camera for taking images of the stream of fiber flocks.

A removal unit for selectively removing entities from the stream of fiber flocks can be arranged on the pneumatic fiber transport conduit downstream of the sensor system in the transport direction.

The hitherto existing systems for monitoring contamination in a stream of fiber flocks only distinguished between the alternatives “contamination” and “no contamination”. The present invention abandoned this binary view on contamination and replaced it by a more sophisticated one. It introduced a two-dimensional event field, thanks to which entities, including contamination, can be handled in a differentiated way. By means of the contamination classification, the stream of fiber flocks can be numerically characterized with regard to various entities therein, and classifications of various streams of fiber flocks can be compared with each other. Also, the removal of contamination from the stream of fiber flocks can be done in a more differentiated way. Some classes of events can remain in the stream of fiber flocks, although they may contain contamination, and only such contamination that would disturb in an envisaged end product can be removed from the stream of fiber flocks.

### BRIEF DESCRIPTION OF THE DRAWINGS

The invention is now explained in closer detail by references to the attached drawings.

FIG. 1 schematically shows a device according to the invention.

FIG. 2 shows graphical representations of event fields with scatter plots.

FIG. 3 shows a flow chart of an embodiment of the method according to the invention.

FIG. 4 shows examples of three different spectral distributions in optical signals obtained from three different types of contaminants.

FIG. 5 shows a block diagram of a system for carrying out an embodiment of the method according to the invention.

### DETAILED DESCRIPTION OF THE PREFERRED EMBODIMENTS

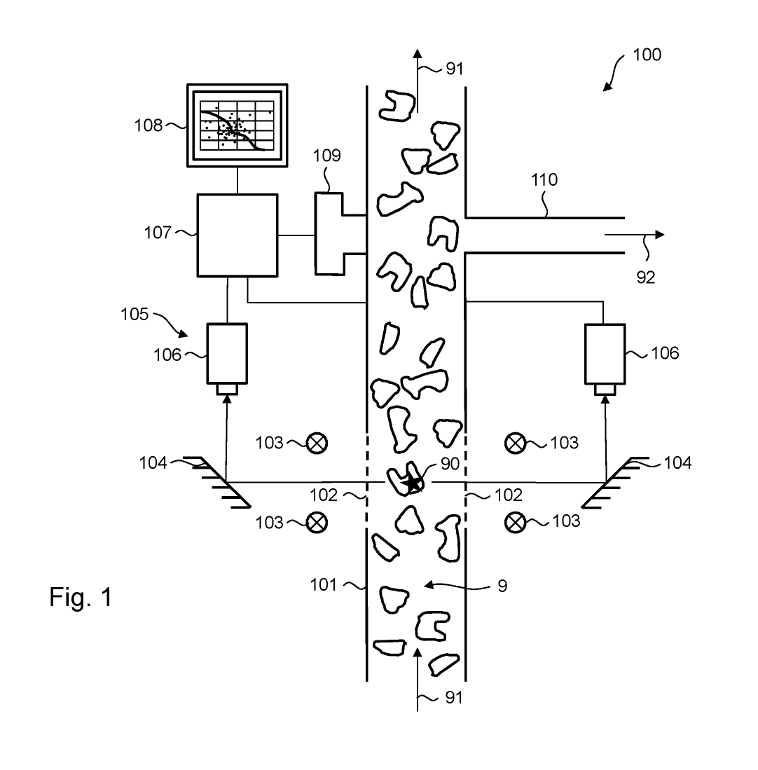

FIG. 1 schematically shows a device 100 according to the invention. The device 100 is for monitoring contamination in a stream of fiber flocks 9. It comprises a pneumatic fiber transport conduit 101 for pneumatically transporting the stream of fiber flocks 9 in an airflow. The transport direction of the stream of fiber flocks 9 and the airflow is indicated in FIG. 1 by arrows 91.

Four light sources 103, such as fluorescent tubes, are arranged in the vicinity of windows 102 in a wall of the fiber transport conduit 101. The light sources 103 illuminate from various directions the stream of fiber flocks 9 in the fiber transport conduit 101.

A sensor system 105 is arranged on the fiber transport conduit 101. It detects characteristics of entities, including contamination, in the stream of fiber flocks 9. In the embodiment of FIG. 1, the sensor system 105 comprises two cameras 106, e.g., CCD cameras, that take images of the stream of fiber flocks 9 through the windows 102 from two different directions. After interaction with the stream of fiber flocks 9, the light can be deflected between the windows 102 and the cameras 106 by means of tilted mirrors 104. It should be understood that the cameras 106 are only an example of a sensor system 105, and that alternative or additional sensor systems can be used in the device 100 according to the invention. Such alternative or additional sensor systems could detect characteristics of entities based on electromagnetic waves other than light, such as microwaves, on acoustic waves, etc. Some of the alternative sensor systems do not need any light sources.

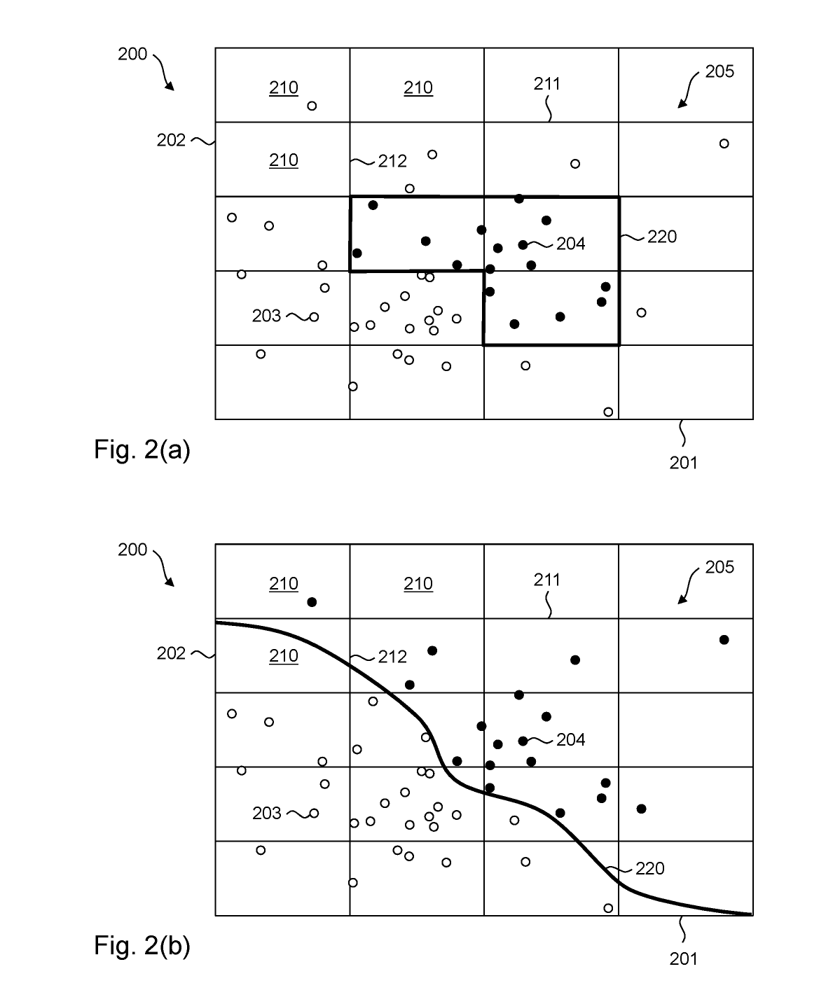

The cameras 106 are connected to an evaluation unit 107 for evaluating output signals of the sensor system 105. The evaluation unit 107 is configured for determining from the output signals of the sensor system 105 values of a first parameter and a second parameter of the entities. The evaluation unit 107 is further configured for providing an event field 200 as discussed below with reference to FIG. 2, and for entering the values of the first parameter and the second parameter determined for an entity in the event field 200 as coordinates of an event 203, 204 representing the entity. The evaluation unit is preferably a computer.

The evaluation unit 107 is connected to an output unit 108 for outputting a result of the evaluation. The output unit 108 is configured for outputting a graphical representation of the event field 200 as discussed below with reference to FIG. 2. The output unit 108 can be, for instance, a display screen or a printer. In one embodiment, it is a touchscreen and thus serves as an input and output unit.

A removal unit 109 for selectively removing entities from the stream of fiber flocks 9 is arranged on the pneumatic fiber transport conduit 101 downstream of the sensor system 105 with regard to the transport direction 91. Such a removal unit 109 is known as such, e.g., from WO-2006/079426 A1. In a preferred embodiment, it comprises a plurality of pressurized air nozzles which are individually controllable by the evaluation unit 107. When the sensor system 105 detects an impermissible contamination 90 in the stream of fiber flocks 9, the appropriate air nozzle of the removal unit 109 is caused to blow pressurized air perpendicularly to the transport direction 91 of the stream of fiber flocks 9 when the contamination 90 has arrived at the removal unit 109. Thus, the contamination 90 is blown out into a removal channel 110 leading away from the fiber transport conduit 101 in a removal direction 92 essentially perpendicular to the transport direction 91 of the stream of fiber flocks 9. The uncontaminated fiber flocks continue on their way with the stream of fiber flocks 9.

The removal unit 109 can be controlled by the evaluation unit 107 and/or directly by the sensor system 105. In the latter case, a microprocessor can be associated with each camera 106, and the cameras 106 can be directly connected with the removal unit 109. Such direct connections are not drawn in FIG. 1 for the sake of simplicity. In a further alternative, the removal unit 109 is controlled by a microprocessor associated with the removal unit 109 itself.

As mentioned above, a graphical representation of the event field 200 provided by the evaluation unit 107 can be output on the output unit 108. Two examples of graphical representations of the event field 200 are shown in FIG. 2. The event field 200 contains a quadrant or a part of a quadrant of a two-dimensional Cartesian coordinate system. A first axis 201, e.g., the abscissa, defines the first parameter and a second axis 202, e.g., the ordinate, defines the second parameter. The first parameter can be related to a geometric characteristic of the entities, and preferably is a length or an area of the entities. The second parameter can be related to an optical characteristic of the entities, and preferably is an intensity of light reflected and/or transmitted by the entities. The values of the first parameter and the second parameter determined for an entity are entered in the event field 200 as coordinates of the entity. Thus, an entity is represented by a graphical symbol 203, 204 such as a dot lying at the location corresponding to its coordinates. Such a representation of an entity in the event field 200 is hereinafter called an “event” 203, 204. A plurality of events 203, 204 constitutes a scatter plot 205 showing the coordinates of the corresponding entities.

According to an embodiment of the invention, a plurality of classes of entities in form of non-overlapping areas 210 in the event field 200 are predetermined. In the example according to FIG. 2, the areas 210 are adjacent rectangles delimited from each other by straight lines 211, 212 parallel to the first axis 201 and the second axis 202, respectively. In the exemplified embodiment of FIG. 2, there are 4×5=20 classes 210; other classifications with other shapes and/or other numbers of classes 210 are possible. An event 203, 204 is classified in one of the at least two classes 210 when the coordinates of the event 203, 204 lie in the corresponding area 210. Events 203, 204 classified in at least one of the at least two classes 210 are counted, and the individual numbers of events 203, 204 counted are output for each of the at least one of the at least two classes 210. The numbers of events 203, 204 counted can be output instead of or in addition to the graphical representation shown in FIG. 2. The classification is helpful in numerically characterizing the contamination contained in the stream of fiber flocks 9.

A removal curve 220 representing a removal limit for contamination can be drawn in in the event field 200 and graphically represented together with the event field 200. The removal limit is predetermined as a criterion for the permissibility or impermissibility of the entities. Entities represented by events 203 with coordinates on the one side of the removal curve 220 are left in the stream of fiber flocks 9, whereas entities represented by events 204 with coordinates on the other side of the removal curve 220 are removed from the stream of fiber flocks 9. Events 203, 204 corresponding to the permissible and impermissible entities, respectively, can be represented by different graphical symbols, such as different shapes, different colors and/or different fillings. In the exemplified embodiment of FIG. 2, permissible events 203 are represented by blank circles, whereas impermissible events 204 are represented by filled circles.

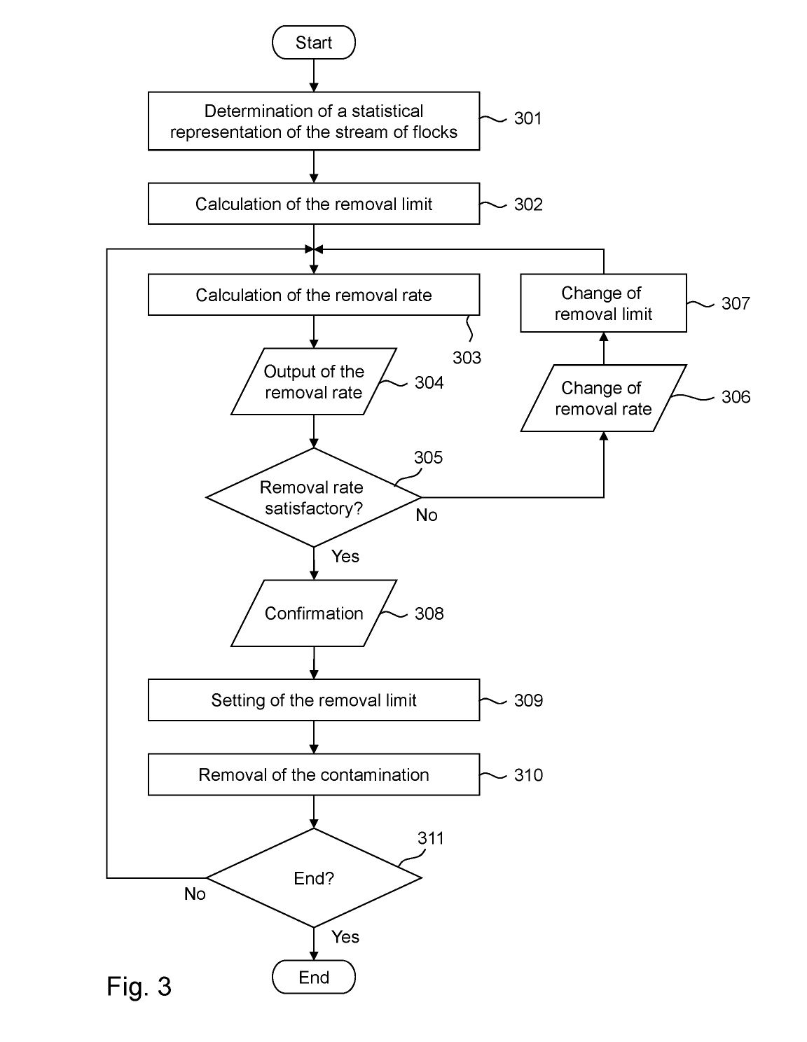

The removal limit can be predetermined by an operator's input, can be taken over from a database containing various types of removal limits, or can be calculated automatically as described below with reference to FIG. 3.

In the embodiment of FIG. 2(a), the removal curve 220 follows the class boundaries 211, 212. Alternatively, the removal curve 220 can be independent of the classes 210 and can thus be defined by an operator in an essentially free manner. An example of the latter alternative is shown in FIG. 2(b).

FIG. 3 shows a flow chart of an embodiment of the method according to the invention for automatically predetermining the removal limit. The calculations in this method are preferably performed by the evaluation unit 107 (see FIG. 1).

The method is started in the simplest possible way. For this purpose a start button is provided, which can be labeled for example with “smart limit”, “auto setup” or the like, on a user interface. The start button can be realized either by hardware or by software. In the latter case it can be displayed symbolically on the output unit 108 (see FIG. 1) and can be actuated by means of an input unit such as a keyboard or a computer mouse, or by contact if there is a touchscreen 108.

A statistical representation of the test material is determined in a calibration process 301. The statistical representation concerns a scatter plot 205 of events 203, 204 as shown in FIG. 2. The statistical representation is preferably obtained by detecting and evaluating a sufficiently large number of entities.

The removal limit and the removal curve 220 as its graphical representation (see FIG. 2) are automatically calculated 302 on the basis of the determined statistical representation. The removal curve 220 can be calculated for example based on a predefined curve shape, which is then fitted into an appropriate position by means of a similarity transformation such as scaling, translation and/or rotation. The position of the removal curve 220 depends on the desired removal rate. In the simplest of cases, the initial removal rate can be a fixedly predetermined value such as 5000 removals per hour for example. The operator can be offered a number of choices for the selection of the initial removal rate, e.g.:
“Low” for a low removal rate (e.g. 1000 removals per hour) or low quality;
“Medium” for a medium removal rate (e.g. 5000 removals per hour) or medium quality, and
“High” for a high removal rate (e.g. 15,000 removals per hour) or high quality.

Selection buttons realized by hardware or software can be provided for selection. For the purpose of complete freedom of selection, the operator can be provided with a possibility of free entry of a desired removal rate.

On the basis of the determined statistical representation and the calculated removal limit, a number of removals relating to time or to the mass of the stream of fiber flocks 9 is calculated automatically 303. This removal rate is obtained from the sum total of all events which are impermissible according to the removal limit.

The operator must be provided with the possibility to provide a comment on the removal rate which follows from the calculated removal limit. For this purpose the removal rate is output 304 on the output unit 108 after its calculation 303. The operator is asked 305 to confirm or change the displayed removal rate. A confirmation button for confirmation of the current removal limit and the removal rate is provided. The removal rate can be changed 306 for instance by means of incremental buttons by an increment, e.g. by 1000 removals per hour. The increment can be proposed or calculated automatically, preferably as a specific fraction, e.g. 20%, of the removal rate. The removal limit is changed automatically 307 as a result of the entered change command for the removal rate. A new removal rate which follows to the changed removal limit is calculated 303. The previously determined statistical representation is used as the basis for this calculation. The operator will be given an opportunity 305 to provide a comment on the new removal rate and to change the same optionally 306 if necessary. The described loop for the optimization of the removal limit or the removal rate can be passed as often until the operator is satisfied and confirms the same 308. The removal limit is only then set 309 so as to be effective for removing contamination 310 from the stream of fiber flocks 9. The setting 309 the removal limit comprises transmitting the removal limit to the unit that controls the removal unit 109, and storing it there. The controlling unit can be the sensor system 105, the evaluation unit 107 and/or the removal unit 109 itself.

It can be advantageous to repeat 311 the process described above periodically or after a major change in the production process. Such a repetition 311 includes a recalculation of the removal rate 303, its output 304 and, if necessary, a change 306 of the removal rate.

A graphical representation of the event field 200, including the scatter plot 205 of events 203, 204, the areas 210 representing the classes of entities and/or the removal curve 220 representing the removal limit is preferably output on the output unit 108 (see FIG. 1).

A classification of contaminants as described with reference to FIG. 2 can be done for each of various types of contaminants. Examples of types of contaminants comprise the following:
(a) Vegetable and other organic contaminants;
(b) White and transparent contaminants; and
(c) Colored contaminants.

Such types of contaminants can be distinguished from each other by determining values of a third parameter of the entities. For each type of contaminants, an event field 200 as discussed with reference to FIG. 2 is provided. Thus, there are separate event fields 200 and/or separate classification results for, e.g., (a) vegetable and other organic contaminants, (b) white and transparent contaminants and (c) colored contaminants, respectively. A criterion related to the third parameter of the entities is assigned to each of the event fields 200. An entity is classified in one of the event fields 200, depending on the fulfillment of the criterion by the value of the at least third parameter determined for said entity. The criterion can depend solely on the third parameter of the entities. Alternatively, it can depend on the third parameter and additionally on one or several further parameters of the entities. For instance, a geometric characteristic of the entities (i.e., the first parameter as discussed with reference to FIG. 2) can be entered into the criterion in addition to the third parameter.

The third parameter can be, for instance, a color of the entities, i.e., a spectral distribution of broadband electromagnetic radiation after interaction with the entities. In this case, the following criteria can be predetermined:
(a) For vegetable and other organic contaminants: the spectral distribution has a peak in the green and/or yellow range (light wavelengths between about 495 nm and 590 nm), and no other significant peak in the visible range.
(b) For white and transparent contaminants: the spectral distribution has values significantly different from zero in the blue range (between about 435 nm and 495 nm), in the green range (between about 495 nm and 570 nm) and in the red range (between about 630 nm and 770 nm).
(c) For colored contaminants: all other cases.

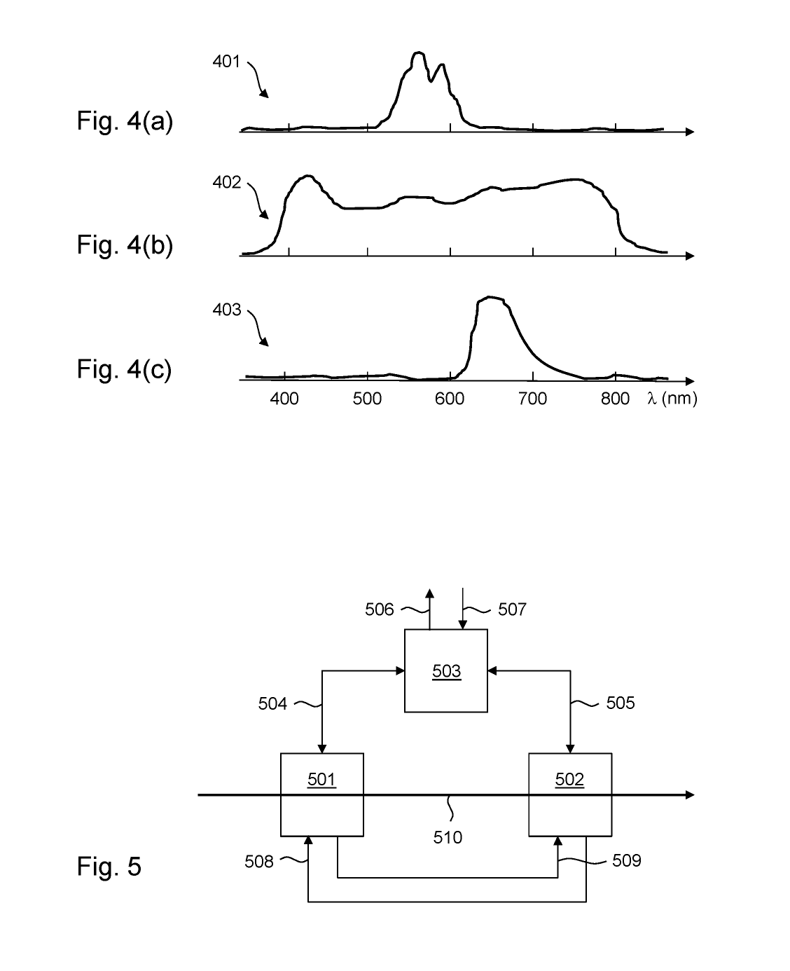

Three examples of different spectral distributions 401-403, as could be determined from optical signals from different entities, are shown in FIG. 4. Each graph 401-403 shows an intensity of light reflected on an entity as a function of the light wavelength X. The spectral distribution 401 of FIG. 4(a) fulfills the above criterion (a); therefore, the corresponding entity would be classified in a first event field for vegetable and organic contaminants. The spectral distribution 402 of FIG. 4(b) fulfills the above criterion (b); therefore, the corresponding entity would be classified in a second event field for white and transparent contaminants. The spectral distribution 403 of FIG. 4(c) fulfills neither the above criterion (a) nor (b); therefore, the corresponding entity is probably a red contaminant and would be classified in a third event field for colored contaminants.

FIG. 5 shows a block diagram of a system 500 for carrying out a further embodiment of the method according to the invention. A horizontal arrow 510 symbolizes a flow of material in a production site such as a spinning mill. The material in the flow 510 can have the same structure, namely, fiber flocks, throughout the whole flow 510, or can change its structure from left to right, e.g., from fiber flocks to a fibrous web, then to a sliver, then to a roving, then to a yarn, etc. Thus, the arrow 510 can encompass the whole textile production chain, all types of textile structures and all types of textile production machines.

A first monitoring device 501 monitors contamination in a stream of fiber flocks, which stream is part of the material flow 510. The first monitoring device 501 is a device 100 according to the invention, as schematically depicted in FIG. 1. A second monitoring device 502 is arranged in the material flow 510 downstream of the first monitoring device 501. The second monitoring device 502 subsequently monitors contamination in the material flow 510, i.e., in the stream of fiber flocks or in an intermediate or a product containing fibers from the stream of fiber flocks monitored by the first monitoring device 501. At the location of the second monitoring device 502, the material flow 510 can still be a stream of fiber flocks. In this case, the second monitoring device 502 can be similar to the first monitoring device 501, depicted as a monitoring device 100 in FIG. 1, except for the removal unit 109, which can be present, but is not necessarily needed in the second monitoring device 502. Alternatively, the second monitoring device 502 can be arranged at a further stage of the textile production chain. It can be, for instance, a yarn clearer with a contamination-clearing capability, arranged on a yarn-winding machine winding yarn containing fibers from the stream of fiber flocks 9 monitored by the first monitoring device 501. Yarn clearers as such are known, e.g., from U.S. Pat. No. 6,244,030 B1.

A control unit 503 is connected via a first connection 504 and a second connection 505 with the first monitoring device 501 and the second monitoring device 502, respectively. The control unit 503 collects data from the first monitoring device 501 and the second monitoring device 502, processes them statistically and outputs reports generated therefrom to an operator, which outputs are indicated in FIG. 5 by an output arrow 506. It also receives inputs from the operator, e.g., requirements with regard to quality, which inputs are indicated in FIG. 5 by an input arrow 507. The control unit 503 can be realized as a computer with corresponding input and/or output peripheral devices. It can also be connected with other devices in the material flow 510, which are, however, not shown in FIG. 5.

According to an embodiment of the invention, the removal limit in the first monitoring device 501 is changed depending on a monitoring result of the second monitoring device 502. Thus, there is a closed control loop controlling the removal of contamination by the first monitoring device 501. The feedback in the closed control loop is indicated in FIG. 5 by a separate arrow 508; however, it can be realized via the connections 504, 505 between the control unit 503 and the monitoring devices 501, 502, which connections 504, 505 are preferably bidirectional. The control unit 503 can act as a controller within the closed control loop. If, for example, the second monitoring device 502 finds too many contaminants in a certain class 210, the control unit 503 can automatically adapt the removal limit in the first monitoring device 501 so as to remove more of the contaminants in said class 210.

According to another embodiment of the invention, the removal limit in the second monitoring device 502 is changed depending on a monitoring result of the first monitoring device 501. In this embodiment, the first monitoring device 501 controls the second monitoring device 502 in an open control loop, which is indicated in FIG. 5 by an arrow 509. The open control loop 509 can be realized via the preferably bidirectional connections 504, 505 between the control unit 503 and the monitoring devices 501, 502. The control unit 503 can act as a controller within the open control loop.

It is understood that the present invention is not limited to the embodiments as discussed above. The person skilled in the art will be able to derive further variants with knowledge of the invention which shall also belong to the subject matter of the present invention.

### LIST OF REFERENCE NUMERALS

100 Device according to the invention
101 Fiber transport conduit
102 Windows in wall of fiber transport conduit
103 Light sources
104 Mirrors
105 Sensor system
106 Cameras
107 Evaluation unit
108 Output unit
109 Removal unit
110 Removal channel
200 Event field
201, 202 First and second axes of the event field
203 Permissible event
204 Impermissible event
205 Scatter plot
210 Areas in the event field representing classes of contaminants
211 Horizontal class boundaries
212 Vertical class boundaries
220 Removal curve
401-403 Spectral distributions
501, 502 First and second monitoring devices
503 Control unit
504, 505 First and second connections
506 Output from the control unit
507 Input into the control unit
508 Feedback in closed control loop
509 Control in open control loop
510 Flow of material
9 Stream of fiber flocks
90 Contaminant
91 Transport direction of the stream of fiber flocks
92 Removal direction

1. A method for monitoring contamination in a stream of fiber flocks transported pneumatically in an airflow, wherein characteristics of entities, including contamination, in the stream of fiber flocks are detected and evaluated, comprising:
determining values of a first parameter and a second parameter of the entities from the characteristics of the entities,
providing an event field, which contains at least a part of a quadrant of a two-dimensional Cartesian coordinate system, wherein a first axis defines the first parameter and a second axis defines the second parameter, and
the determined values of the first parameter and the second parameter for an entity are entered in the event field as coordinates of an event representing the entity.

2. The method according to claim 1, wherein:
the first parameter is related to a geometric characteristic of the entities, and
the second parameter is related to an optical characteristic of the entities.

3. The method according to claim 1, wherein:
at least two classes of entities in form of non-overlapping areas in the event field are predetermined, and
an entity is classified in one of the at least two classes when the coordinates of an event representing the entity lie in the corresponding area.

4. The method according to claim 3, wherein entities classified in at least one of the at least two classes are counted and individual numbers of entities counted are output for each of the at least one of the at least two classes.

5. The method according to claim 1, wherein:
at least two event fields are provided, each of the at least two event fields containing at least a part of a quadrant of a two-dimensional Cartesian coordinate system, wherein a first axis defines the first parameter and a second axis defines the second parameter,
in each of the at least two event fields at least two classes of entities in form of non-overlapping areas in the event field are predetermined,
a criterion related to at least a third parameter of entities is assigned to each of the at least two event fields,
values of the least a third parameter are determined from the characteristics of the entities, and
an entity is classified in one of the at least two event fields, depending on the fulfillment of the criterion by the value of the at least third parameter determined for said entity.

6. The method according to claim 5, wherein the third parameter is related to an optical characteristic of the entities.

7. The method according to claim 1, wherein:
a removal limit in form of a removal curve in the event field is predetermined as a criterion for the permissibility or impermissibility of the entities, and
entities represented by events with coordinates on one side of the removal curve are left in the stream of fiber flocks, whereas entities represented by events with coordinates on another side of the removal curve are removed from the stream of fiber flocks.

8. The method according to claim 1, wherein:
at least two classes of entities in form of non-overlapping areas in the event field are predetermined, and an entity is classified in one of the at least two classes when the coordinates of an event representing the entity lie in the corresponding area,
a removal limit in form of a removal curve in the event field is predetermined as a criterion for the permissibility or impermissibility of the entities, and entities represented by events with coordinates on one side of the removal curve are left in the stream of fiber flocks, whereas entities represented by events with coordinates on another side of the removal curve are removed from the stream of fiber flocks, and
the event field, including a scatter plot showing coordinates of events representing entities, the removal curve, and the at least two areas are graphically represented.

9. The method according to claim 8, wherein:
a statistical representation of the stream of fiber flocks is determined from the values of the first parameter and the second parameter of the entities,
a removal limit is predetermined on the basis of the statistical representation,
at least one of a time-related and a mass-related number of impermissible entities is calculated from the statistical representation and the removal limit,
at least one of the time-related and mass-related number of impermissible entities is output on an output unit,
an operator is requested to enter a comment on the output time-related or mass-related number of impermissible entities by means of an input unit, and
the removal limit is set automatically according to the entered comment.

10. The method according to claim 8, wherein a second monitoring of contamination is subsequently performed in at least one of the stream of fiber flocks and a product containing fibers from the stream of fiber flocks, and the removal limit in the monitoring of contamination in the stream of fiber flocks is changed depending on a result of the second monitoring of contamination.

11. The method according to claim 8, wherein a second monitoring of contamination is subsequently performed in at least one of the stream of fiber flocks and a product containing fibers from the stream of fiber flocks, and a removal limit in the second monitoring of contamination is changed depending on a result of the monitoring of contamination in the stream of fiber flocks.

12. The method according to claim 11, wherein the second monitoring is performed by means of a yarn clearer in a yarn containing fibers from the stream of fiber flocks.

13. A device for monitoring contamination in a stream of fiber flocks, comprising:
a pneumatic fiber transport conduit for transporting the stream of fiber flocks,
a sensor system for detecting characteristics of entities, including contamination, in the stream of fiber flocks, the sensor system being arranged on the pneumatic fiber transport conduit, and
an evaluation unit for evaluating output signals of the sensor system, the evaluation unit configured for,
determining from the output signals of the sensor system values of a first parameter and a second parameter of the entities,
providing an event field, which contains a quadrant or a part of a quadrant of a two-dimensional Cartesian coordinate system, wherein a first axis defines the first parameter and a second axis defines the second parameter, and
entering the values of the first parameter and the second parameter determined for an entity in the event field as coordinates of an event representing the entity.

14. The device according to claim 13, wherein the device further comprises an output unit for outputting a result of the evaluation, the output unit being configured for outputting a graphical representation of the event field, including a scatter plot showing the coordinates of events representing entities.

15. The device according to claim 13, wherein the sensor system comprises a camera for taking images of the stream of fiber flocks.

16. The device according to claim 13, further comprising a removal unit for selectively removing entities from the stream of fiber flocks is disposed on the pneumatic fiber transport conduit downstream of the sensor system.
